(19) United States
(12) Patent Application Publication
(10) Pub. No.: US 2021/0358640 A1
(43) Pub. Date: Nov. 18, 2021

(54) MACHINE LEARNING MODELS FOR MULTI-RISK-LEVEL DISEASE SPREAD FORECASTING

(71) Applicant: Optum Technology, Inc., Eden Prairie, MN (US)

(72) Inventors: Kartik Chaudhary, Bangalore (IN); Vineet Shukla, Bangalore (IN); Pooja Mahesh Rajdev, Bangalore (IN); V Kishore Ayyadevara, Hyderabad (IN); Shivam Mishra, New Delhi (IN); Neelesh Bhushan, Gurugram (IN); Sahil Jolly, Ghaziabad (IN)

(21) Appl. No.: 17/002,232

(22) Filed: Aug. 25, 2020

(30) Foreign Application Priority Data
May 18, 2020 (IN) 202011020854

(51) Int. Cl. G16H 50/80; G06N 20/00; G16H 50/20

(52) U.S. Cl. CPC G16H 50/80; G06Q 10/04; G16H 50/20; G06N 20/00

(57) ABSTRACT

There is a need for more reliable and efficient disease spread forecasting. This need can be addressed by, for example, solutions for performing optimization-based disease spread forecasting using a multi-risk-level disease spread forecasting machine learning model. In one example, a method includes identifying a retrospective timeseries data object; processing the retrospective timeseries data object to generate a plurality of temporally dynamic parameters of the multi-risk-level disease spread forecasting machine learning model, wherein the plurality of temporally dynamic parameters comprise a general susceptibility parameter, a heightened risk ratio parameter, a heightened risk infection probability parameter, and a non-heightened risk infection probability parameter; and subsequent to generating the plurality of temporally dynamic parameters, enabling access to the multi-risk-level disease spread forecasting machine learning model to generate a prospective disease spread forecast data object and perform one or more prediction-based actions based on the prospective disease spread forecast data object.

### CROSS-REFERENCES TO RELATED APPLICATIONS

The present application claims priority to Indian Provisional Patent Application No. 202011020854 (filed on May 18, 2020), which is incorporated herein by reference in its entirety.

### BACKGROUND

Various embodiments of the present invention address technical challenges related to performing disease spread forecasting. Various embodiments of the present invention address the shortcomings of existing disease spread forecasting systems and disclose various techniques for efficiently and reliably performing disease spread forecasting.

### BRIEF SUMMARY

In general, embodiments of the present invention provide methods, apparatus, systems, computing devices, computing entities, and/or the like for performing optimization-based disease spread forecasting using a multi-risk-level disease spread forecasting machine learning model, where the multi-risk-level disease spread forecasting machine learning model is characterized by a group of temporally dynamic parameters, and where the group of temporally dynamic parameters comprise a general susceptibility parameter, a heightened risk ratio parameter, a heightened risk infection probability parameter, and a non-heightened risk infection probability parameter.

In accordance with one aspect, a method is provided. In one embodiment, the method comprises: identifying a retrospective timeseries data object; processing the retrospective timeseries data object to generate a plurality of temporally dynamic parameters of the multi-risk-level disease spread forecasting machine learning model, wherein the plurality of temporally dynamic parameters comprise a general susceptibility parameter, a heightened risk ratio parameter, a heightened risk infection probability parameter, and a non-heightened risk infection probability parameter; and subsequent to generating the plurality of temporally dynamic parameters, enabling access to the multi-risk-level disease spread forecasting machine learning model to generate a prospective disease spread forecast data object and perform one or more prediction-based actions based at least in part on the prospective disease spread forecast data object.

In accordance with another aspect, a computer program product is provided. The computer program product may comprise at least one computer-readable storage medium having computer-readable program code portions stored therein, the computer-readable program code portions comprising executable portions configured to: identify a retrospective timeseries data object; process the retrospective timeseries data object to generate a plurality of temporally dynamic parameters of the multi-risk-level disease spread forecasting machine learning model, wherein the plurality of temporally dynamic parameters comprise a general susceptibility parameter, a heightened risk ratio parameter, a heightened risk infection probability parameter, and a non-heightened risk infection probability parameter; and subsequent to generating the plurality of temporally dynamic parameters, enable access to the multi-risk-level disease spread forecasting machine learning model to generate a prospective disease spread forecast data object and perform one or more prediction-based actions based at least in part on the prospective disease spread forecast data object.

In accordance with yet another aspect, an apparatus comprising at least one processor and at least one memory including computer program code is provided. In one embodiment, the at least one memory and the computer program code may be configured to, with the processor, cause the apparatus to: identify a retrospective timeseries data object; process the retrospective timeseries data object to generate a plurality of temporally dynamic parameters of the multi-risk-level disease spread forecasting machine learning model, wherein the plurality of temporally dynamic parameters comprise a general susceptibility parameter, a heightened risk ratio parameter, a heightened risk infection probability parameter, and a non-heightened risk infection probability parameter; and subsequent to generating the plurality of temporally dynamic parameters, enable access to the multi-risk-level disease spread forecasting machine learning model to generate a prospective disease spread forecast data object and perform one or more prediction-based actions based at least in part on the prospective disease spread forecast data object.

### BRIEF DESCRIPTION OF THE DRAWINGS

Having thus described the invention in general terms, reference will now be made to the accompanying drawings, which are not necessarily drawn to scale, and wherein:

FIG. 1 provides an exemplary overview of an architecture that can be used to practice embodiments of the present invention.

FIG. 2 provides an example predictive data analysis computing entity in accordance with some embodiments discussed herein.

FIG. 3 provides an example external computing entity in accordance with some embodiments discussed herein.

FIG. 4 is a flowchart diagram of an example process for performing optimization-based disease spread forecasting using a multi-risk-level disease spread forecasting machine learning model in accordance with some embodiments discussed herein.

FIG. 5 provides an operational example of the operations and parameters of a multi-risk-level disease spread forecasting machine learning model in accordance with some embodiments discussed herein.

FIG. 6 is a flowchart diagram of an example process for generating a multi-risk-level disease spread forecasting machine learning model in accordance with some embodiments discussed herein.

FIG. 7 provides an operational example of generating an optimized susceptibility ratio parameter in accordance with some embodiments discussed herein.

FIG. 8 provides an operational example of a prediction output user interface in accordance with some embodiments discussed herein.

### DETAILED DESCRIPTION

Various embodiments of the present invention now will be described more fully hereinafter with reference to the accompanying drawings, in which some, but not all, embodiments of the inventions are shown. Indeed, these inventions may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. The terms “illustrative” and “exemplary” are used to be examples with no indication of quality level. Like numbers refer to like elements throughout. Moreover, while certain embodiments of the present invention are described with reference to predictive data analysis, one of ordinary skill in the art will recognize that the disclosed concepts can be used to perform other types of data analysis.

### I. Overview

One innovative and technologically advantageous aspect of the present invention relates to generating reliable disease spread forecasts (e.g., pandemic spread forecasts) by using machine learning techniques that integrate predictive insights about varying levels of risk among susceptible population. For example, various embodiments of the present invention disclose a multi-risk-level machine learning model that integrates predictive insights varying levels of risk between essential workers, ordinary population, and quarantined population during a pandemic. Such a machine learning model is capable of capturing the effects of various levels of societal opening policies as well as various measures to quarantine infected population on the overall trajectory of the disease spread. By disclosing the techniques for generating reliable disease spread forecasts (e.g., pandemic spread forecasts) that rely on using machine learning techniques seeking to integrate predictive insights about varying levels of risk among susceptible population, various embodiments of the present invention improve the reliability of predictive data analysis solutions configured to perform disease spread forecasting and make important technical contributions to the fields of predictive data analysis and disease spread forecasting (e.g., pandemic forecasting).

Another innovative and technologically advantageous aspect of the present invention relates to generating reliable disease spread forecasts (e.g., pandemic spread forecasts) by using machine learning techniques that integrate predictive insights about varying exposure/transition probabilities across time by using temporally dynamic parameters. For example, in some embodiments, a machine learning model configured to perform disease spread forecasting has a group of temporally dynamic parameters that are updated over time based at least in part on incoming data. Examples of such parameters include a heightened risk growth parameter, which is also referred to herein as λ or lambda; a heightened risk infection probability parameter, which is also referred to herein as γ or gamma; a containment probability parameter, which is also referred to herein as Θ or theta; and a termination probability parameter, which is also referred to herein as ρ or rho. By utilizing the techniques for generating reliable disease spread forecasts (e.g., pandemic spread forecasts) that rely on using machine learning techniques that integrate predictive insights about varying exposure/transition probabilities across time via using temporally dynamic parameters, various embodiments of the present invention further improve the reliability of predictive data analysis solutions configured to perform disease spread forecasting and make important technical contributions to the fields of predictive data analysis and disease spread forecasting (e.g., pandemic forecasting).

A yet another innovative and technologically advantageous aspect of the present invention relates to generating reliable disease spread forecasts (e.g., pandemic spread forecasts) by using machine learning techniques that utilize a retrospective timeseries data object to calculate error measures that can in turn be used to determine inferred susceptibility parameters, rather than constant susceptibility parameters. For example, in some embodiments, determining the general susceptibility parameter comprises identifying a plurality of candidate general susceptibility parameter values; for each candidate general susceptibility parameter value of the plurality of candidate general susceptibility parameter values, generating a mean absolute error measure; and determining the general susceptibility parameter based at least in part on each mean absolute error measure for a candidate general susceptibility parameter value of the plurality of candidate general susceptibility parameter values. In some of the noted embodiments, a predictive data analysis computing entity may fix the values of the non-optimizable parameters of the multi-risk-level disease spread forecasting machine learning model, then may proceed to generate a mean squared error (MAE) measure for each candidate value of the general susceptibility parameter in order to generate one or more target forecasts, and then compare the target forecasts to ground-truth data provided by historical data in order to determine an optimal value of the general susceptibility parameter, such as the least value of the general susceptibility parameter that causes the MAE graph to have a slope of zero or near-zero (e.g., within a 0.001 range of zero). By utilizing techniques for generating reliable disease spread forecasts (e.g., pandemic spread forecasts) that rely on using machine learning techniques that in turn utilize computed error measures to determine optimal values of inferred susceptibility parameters, various embodiments of the present invention further improve the reliability of predictive data analysis solutions that are configured to perform disease spread forecasting and make important technical contributions to the fields of predictive data analysis and disease spread forecasting (e.g., pandemic forecasting).

### II. Definitions

The term “retrospective timeseries data object” may refer to a data construct that is configured to describe one or more observed metrics related to disease spread across one or more time units. For example, the retrospective timeseries data object may describe observed metrics about at least one of currently infected cases (aka. active cases) of a disease across one or more time units (e.g., one or more days), recovered cases of a disease across one or more time units, and deceased cases (aka. terminated cases) of a disease across one or more time units. In some embodiments, the retrospective timeseries data object describe one or more observed metrics about disease spread for a target period of time, such as a latest recorded time unit (i.e., a latest recorded time unit, such as a current day). Examples of such observed metrics may include one or more of the following: retrospective susceptibility count (Sₜ) values, retrospective containment count (Qₜ) values, retrospective recovery count (Rₜ) values, retrospective termination count (Dₜ) values, retrospective infection count (Iₜ) values, retrospective heightened risk count values (Eₜ), retrospective non-heightened risk count values (Nₜ), and a total population count (N).

The term “retrospective susceptibility count,” which is also referred to herein as Sₜ, may refer to a data construct that is configured to describe a number of individuals deemed to be susceptible to a particular disease during a target time period t, such as during a current time period (e.g., during a current day). As described above, in some embodiments, Sₜ may be described by a retrospective timeseries data object. In some other embodiments, Sₜ may be estimated based at least in part on a total population count (N) at the target period, a general susceptibility parameter (α or alpha), a retrospective recovery count (Rₜ), and a recovered susceptibility parameter (α′ or alpha′).

The term “retrospective heightened risk count,” which is also referred to herein as Eₜ, may refer to a data construct that is configured to describe a number of individuals deemed to be having a higher level of risk of disease spread relative to the normal population during a target time period t, such as during a current time period (e.g., during a current day). This may include essential workers, healthcare workers, people with physiological conditions that are deemed to be associated with heightened risk of disease spread, and/or the like. As described above, in some embodiments, Eₜ may be described by a retrospective timeseries data object. In some other embodiments, Eₜ may be estimated based at least in part on a retrospective susceptibility count (Sₜ) and a heightened risk ratio parameter (λ or lambda).

The term “retrospective non-heightened risk count,” which is also referred to herein as Nₜ, may refer to a data construct that is configured to describe a number of individuals deemed to be having a risk of disease spread that is equivalent to the risk of disease spread of the normal population during a target time period t, such as during a current time period (e.g., during a current day). This may include grocery store shoppers and/or retail shoppers. As described above, in some embodiments, Nₜ may be described by a retrospective timeseries data object. In some other embodiments, Nₜ may be estimated based at least in part on a retrospective susceptibility count (Sₜ) and a non-heightened risk ratio parameter (1-λ or 1-lambda).

The term “retrospective infection count,” which is also referred to herein as Iₜ, may refer to a data construct that is configured to describe a number of individuals deemed to be currently infected by a disease during a target time period t, such as during a current time period (e.g., during a current day). As described above, in some embodiments, Iₜ may be described by a retrospective timeseries data object, and may for example be determined based at least in part on the number of active cases for the target time period as described by the retrospective timeseries data object. In some other embodiments, Iₜ may be estimated based at least in part on a retrospective heightened risk count (Eₜ), a heightened risk infection probability parameter (y or gamma), a retrospective non-heightened risk count (Nₜ), and a non-heightened risk infection probability parameter (β or Beta).

The term “retrospective containment count,” which is also referred to herein as Qₜ, may refer to a data construct that is configured to describe a number of individuals that are deemed infected but are also deemed to be at a minimal risk of disease spread due to physical containment of those individuals during a target time period t, such as during a current time period (e.g., during a current day). This may include quarantined individuals, individuals under lockdown measures, individuals under stay-at-home measures, individuals under curfew measures, and/or the like. As described above, in some embodiments, Qₜ may be described by a retrospective timeseries data object. In some other embodiments, Qₜ may be estimated based at least in part on a retrospective infection count (Iₜ) and a containment probability parameter (Θ or theta).

The term “retrospective termination count,” which is also referred to herein as Dₜ, may refer to a data construct that is configured to describe a number of individuals deemed to be eliminated from the total population as a result of the disease. This may include deceased individuals, brain-dead individuals, and/or the like. As described above, in some embodiments, Dₜ may be described by a retrospective timeseries data object. In some other embodiments, Dₜ may be estimated based at least in part on a retrospective containment count (Qₜ) and a termination probability parameter (ρ or rho).

The term “retrospective recovery count,” which is also referred to herein as Rₜ, may refer to a data construct that is configured to describe a number of individuals deemed to have been infected with the disease and subsequently recovered from the disease. As described above, in some embodiments, Rₜ may be described by a retrospective timeseries data object. In some other embodiments, Rₜ may be estimated based at least in part on a retrospective containment count (Qₜ) and a recovery probability parameter (δ or delta).

The term “multi-level disease spread forecasting machine learning model” may refer to a data construct that is configured to describe parameters, defined operations, and/or hyper-parameters of a machine learning model (e.g., a statistical model with one or more trained parameters, a model using a system of differential equations with one or more configurable parameters, and/or the like) that generates a disease spread forecast using predictive insights that relate to differing exposure to disease spread among at least two categories of the susceptible population. As one example, a multi-level disease spread forecasting machine learning model may integrate predictive insights about differing exposure to disease spread among a heightened risk portion of the susceptible population (e.g., an essential worker segment of the susceptible population) and a non-heightened risk portion of the susceptible population (e.g., a retail shopper segment of the susceptible population). As another example, a multi-level disease spread forecasting machine learning model may integrate predictive insights about differing exposure to disease spread among a heightened risk portion of the susceptible population (e.g., an essential worker segment of the susceptible population), a non-heightened risk portion of the susceptible population (e.g., a retail shopper segment of the susceptible population), and a minimal-risk portion of the susceptible population (e.g., a quarantined segment of the susceptible population). As yet another example, a multi-level disease spread forecasting machine learning model may integrate predictive insights about differing exposure to disease spread among a heightened risk portion of the susceptible population (e.g., a segment of the susceptible population that includes essential workers working in contained spaces), a non-heightened risk portion of the susceptible population (e.g., a retail shopper segment of the susceptible population), and a medium risk portion of the susceptible population (e.g., a segment of the susceptible population that includes essential workers working in open spaces). As a further example, a multi-level disease spread forecasting machine learning model may integrate predictive insights about differing exposure to disease spread among a heightened risk portion of the susceptible population (e.g., a segment of the susceptible population that includes essential workers working in contained spaces), a non-heightened risk portion of the susceptible population (e.g., a retail shopper segment of the susceptible population), a medium risk portion of the susceptible population (e.g., a segment of the susceptible population that includes essential workers working in open spaces), and a minimal-risk portion of the susceptible population (e.g., a quarantined segment of the susceptible population).

The term “general susceptibility parameter,” which is also referred to herein as a or alpha, may refer to a data construct that is configured to describe an estimated likelihood that a member of a population may be susceptible to disease spread, e.g., an expected/estimated/measured ratio of a total population that is likely to be susceptible to disease spread. In some embodiments, α is a temporally dynamic parameter, i.e., α is a parameter that is updated based at least in part on newly arriving data. For example, in some embodiments, α may be determined in accordance with an optimization technique configured to minimize a measure of error between disease spread forecasts across historical data and ground-truth disease spread metrics determined using the historical data.

The term “recovered susceptibility parameter,” which is also referred to herein as α′ or alpha′, may refer to a data construct that is configured to describe an estimated likelihood that a member of a recovered population may be susceptible to disease spread, e.g., an expected/estimated/measured ratio of the recovered population that is likely to still be susceptible to disease spread. In some embodiments, α′ is determined based at least in part on scientific literature and/or analytical studies. In some embodiments, α′ is expected to be lower than α. In some embodiments, α′may be determined in accordance with an optimization technique configured to minimize a measure of error between disease spread forecasts across historical data and ground-truth disease spread metrics determined using the historical data. In some embodiments, α′ is a temporally dynamic parameter, i.e., α′ is a parameter that is updated based at least in part on newly arriving data.

The term “heightened risk growth parameter,” which is also referred to herein as λ or lambda, may refer to a data construct that is configured to describe an estimated likelihood that a member of a susceptible population may be part of a heightened risk segment of the susceptible population, such as an essential worker segment of the susceptible population. For example, λ may describe a heightened risk ratio of a susceptible population. In some embodiments, λ is determined based at least in part on preexisting data, such as economic data. In some embodiments, λ may be determined in accordance with an optimization technique configured to minimize a measure of error between disease spread forecasts across historical data and ground-truth disease spread metrics determined using the historical data. In some embodiments, λ is a temporally dynamic parameter, i.e., λ is a parameter that is updated based at least in part on newly data.

The term “non-heightened risk growth parameter,” which is also referred to herein as 1-λ or 1-lambda, may refer to a data construct that is configured to describe an estimated likelihood that a member of a susceptible population may be part of a non-heightened risk segment of the susceptible population, such as a regular retail worker segment of the susceptible population. For example, 1-λ may describe a non-heightened risk ratio of a susceptible population. In some embodiments, 1-λ is determined based at least in part on preexisting data, such as economic data. In some embodiments, 1-λ may be determined in accordance with an optimization technique configured to minimize a measure of error between disease spread forecasts across historical data and ground-truth disease spread metrics determined using the historical data. In some embodiments, 1-λ is a temporally dynamic parameter, i.e., 1-λ is a parameter that is updated based at least in part on newly data and/or based at least in part on updates to λ across time.

The term “heightened risk infection probability parameter,” which is also referred to herein as γ or gamma, may refer to a data construct that is configured to describe an estimated likelihood that a member of a heightened risk population may be infected to a disease. For example, γ may describe a historical ratio of heightened risk individuals (e.g., essential works) who have been infected with the disease. In some embodiments, γ may be determined in accordance with an optimization technique configured to minimize a measure of error between disease spread forecasts across historical data and ground-truth disease spread metrics determined using the historical data. In some embodiments, γ is a temporally dynamic parameter, i.e., γ is a parameter that is updated based at least in part on newly data.

The term “non-heightened risk infection probability parameter,” which is also referred to herein as β or beta, may refer to a data construct that is configured to describe an estimated likelihood that a member of a non-heightened risk population may be infected to a disease. For example, β may describe a historical ratio of non-heightened risk individuals (e.g., retail shoppers) who have been infected with the disease. In some embodiments, β may be determined in accordance with an optimization technique configured to minimize a measure of error between disease spread forecasts across historical data and ground-truth disease spread metrics determined using the historical data. In some embodiments, β is a temporally dynamic parameter, i.e., β is a parameter that is updated based at least in part on newly data.

The term “containment probability parameter,” which is also referred to herein as Θ or theta, may refer to a data construct that is configured to describe an estimated likelihood that a member of the infected population may be contained, e.g., a ratio of the infected individuals that are estimated to have been quarantined. In some embodiments, Θ is determined based at least in part on predefined data, e.g., social studies data. In some embodiments, Θ may be determined in accordance with an optimization technique configured to minimize a measure of error between disease spread forecasts across historical data and ground-truth disease spread metrics determined using the historical data. In some embodiments, Θ is a temporally dynamic parameter, i.e., θ is a parameter that is updated based at least in part on newly data.

The term “termination probability parameter,” which is also referred to herein as ρ or rho, may refer to a data construct that is configured to describe an estimated likelihood that a member of the quarantined population may decease/terminate, e.g., a ratio of the quarantined individuals that are estimated to have been deceased. In some embodiments, ρ is determined based at least in part on preexisting data, e.g., death statistics data. In some embodiments, ρ may be determined in accordance with an optimization technique configured to minimize a measure of error between disease spread forecasts across historical data and ground-truth disease spread metrics determined using the historical data. In some embodiments, ρ is a temporally dynamic parameter, i.e., ρ is a parameter that is updated based at least in part on newly data.

The term “recovery probability parameter,” which is also referred to herein as δ or delta, may refer to a data construct that is configured to describe an estimated likelihood that a member of the quarantined population may recover, e.g., a ratio of the quarantined individuals that are estimated to have been recovered. In some embodiments, δ is determined based at least in part on preexisting data, e.g., recovery statistics data, hospitalization data, and/or the like. In some embodiments, δ may be determined in accordance with an optimization technique configured to minimize a measure of error between disease spread forecasts across historical data and ground-truth disease spread metrics determined using the historical data. In some embodiments, δ is a temporally dynamic parameter, i.e., δ is a parameter that is updated based at least in part on newly data.

Term “disease spread forecast data object” may refer to a data construct that is configured to describe at least one or more forecasted/predicted metrics associated with spread of a disease during one or more prospective time periods. For example, the disease spread forecast data object may describe forecasted/predicted metrics associated with spread of a disease during a subsequent day/week/month. Examples of data entries that may be described by a disease spread forecast data object include a prospective susceptibility forecast

$$( \frac{dS ( t )}{dt} ) ,$$

a prospective heightened risk forecast

$$( \frac{dE ( t )}{dt} ) ,$$

a prospective non-heightened risk forecast

$$( \frac{dN ( t )}{dt} ) ,$$

a prospective infection forecast

$$( \frac{dI ( t )}{dt} ) ,$$

a prospective containment forecast

$$( \frac{dQ ( t )}{dt} ) ,$$

a prospective recovery forecast

$$( \frac{dR ( t )}{dt} ) ,$$

and a prospective termination forecast

$$( \frac{dD ( t )}{dt} ) .$$

The term “prospective susceptibility forecast,” which is also referred to herein as

$$( \frac{dS ( t )}{dt} ) ,$$

may refer to a data construct that is configured to describe a predicted change in the quantity of a susceptible population between a current time period and a future time period. In some embodiments, the prospective susceptibility forecast may be determined based at least in part on: (i) the retrospective infection count value (Iₜ), (ii) the retrospective susceptibility count value (Sₜ), (iii) the heightened risk ratio parameter (λ), (iv) the heightened risk infection probability parameter (γ), (v) the non-heightened risk infection probability parameter (β), and (vi) the general susceptibility parameter (α).

The term “prospective heightened risk forecast,” which is also referred to herein as

$$( \frac{dE ( t )}{dt} ) ,$$

may refer to a data construct that is configured to describe a predicted change in the quantity of a heightened risk population (e.g., an essential worker population) between a current time period and a future time period. In some embodiments, the prospective heightened risk forecast may be determined based at least in part on: the prospective susceptibility forecast

$$( \frac{dS ( t )}{dt} ) ,$$

the retrospective heightened risk count value (Eₜ), and the heightened risk infection probability parameter (γ).

The term “prospective non-heightened risk forecast,” which is also referred to herein as

$$( \frac{dN ( t )}{dt} ) ,$$

may refer to a data construct that is configured to describe a predicted change in the quantity of a non-heightened risk population (e.g., a retail shopper population) between a current time period and a future time period. In some embodiments, the prospective non-heightened risk forecast may be determined based at least in part on: the prospective susceptibility forecast

$$( \frac{dS ( t )}{dt} ) ,$$

the retrospective non-heightened risk count value (Nₜ), and the non-heightened risk infection probability parameter (β).

The term “prospective infection forecast,” which is also referred to herein as

$$\frac{dI ( t )}{dt} ,$$

may refer to a data construct that is configured to describe a predicted change in the quantity of an infected population between a current time period and a future time period. In some embodiments, the prospective infection forecast may be determined based at least in part on: the prospective susceptibility forecast

$$( \frac{dS ( t )}{dt} ) ,$$

the retrospective infection count value (Iₜ), and the containment probability parameter (Θ).

The term “prospective containment forecast,” which is also referred to herein as

$$\frac{dQ ( t )}{dt} ,$$

may refer to a data construct that is configured to describe a predicted change in the quantity of an infected but contained (e.g., quarantined) population between a current time period and a future time period. In some embodiments, the prospective contained forecast may be determined based at least in part on: (i) the respective infection count (Iₜ), (ii) the respective containment count (Qₜ), (iii) the containment probability parameter (Θ), (iv) the recovery probability parameter (δ), and (v) the termination probability parameter (ρ).

The term “prospective recovery forecast,” which is also referred to herein as

$$\frac{dR ( t )}{dt} ,$$

may refer to a data construct that is configured to describe a predicted change in the quantity of an infected but recovered population between a current time period and a future time period. In some embodiments, the prospective recovery forecast may be determined based at least in part on the respective containment count (Qₜ) and the recovery probability parameter (δ).

The term “prospective termination forecast,” which is also referred to herein as

$$\frac{dD ( t )}{dt} ,$$

may refer to a data construct that is configured to describe a predicted change in the quantity of an infected but terminated (e.g., deceased) population between a current time period and a future time period. In some embodiments, the prospective recovery termination may be determined based at least in part on the respective containment count (Qₜ) and the termination probability parameter (ρ).

### III. Computer Program Products, Methods, and Computing Entities

Embodiments of the present invention may be implemented in various ways, including as computer program products that comprise articles of manufacture. Such computer program products may include one or more software components including, for example, software objects, methods, data structures, or the like. A software component may be coded in any of a variety of programming languages. An illustrative programming language may be a lower-level programming language such as an assembly language associated with a particular hardware architecture and/or operating system platform. A software component comprising assembly language instructions may require conversion into executable machine code by an assembler prior to execution by the hardware architecture and/or platform. Another example programming language may be a higher-level programming language that may be portable across multiple architectures. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together such as, for example, in a particular directory, folder, or library. Software components may be static (e.g., pre-established or fixed) or dynamic (e.g., created or modified at the time of execution).

A computer program product may include a non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media (including volatile and non-volatile media).

In one embodiment, a non-volatile computer-readable storage medium may include a floppy disk, flexible disk, hard disk, solid-state storage (SSS) (e.g., a solid state drive (SSD), solid state card (SSC), solid state module (SSM), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In one embodiment, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

As should be appreciated, various embodiments of the present invention may also be implemented as methods, apparatus, systems, computing devices, computing entities, and/or the like. As such, embodiments of the present invention may take the form of an apparatus, system, computing device, computing entity, and/or the like executing instructions stored on a computer-readable storage medium to perform certain steps or operations. Thus, embodiments of the present invention may also take the form of an entirely hardware embodiment, an entirely computer program product embodiment, and/or an embodiment that comprises combination of computer program products and hardware performing certain steps or operations. Embodiments of the present invention are described below with reference to block diagrams and flowchart illustrations. Thus, it should be understood that each block of the block diagrams and flowchart illustrations may be implemented in the form of a computer program product, an entirely hardware embodiment, a combination of hardware and computer program products, and/or apparatus, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some exemplary embodiments, retrieval, loading, and/or execution may be performed in parallel such that multiple instructions are retrieved, loaded, and/or executed together. Thus, such embodiments can produce specifically-configured machines performing the steps or operations specified in the block diagrams and flowchart illustrations. Accordingly, the block diagrams and flowchart illustrations support various combinations of embodiments for performing the specified instructions, operations, or steps.

### IV. Exemplary System Architecture

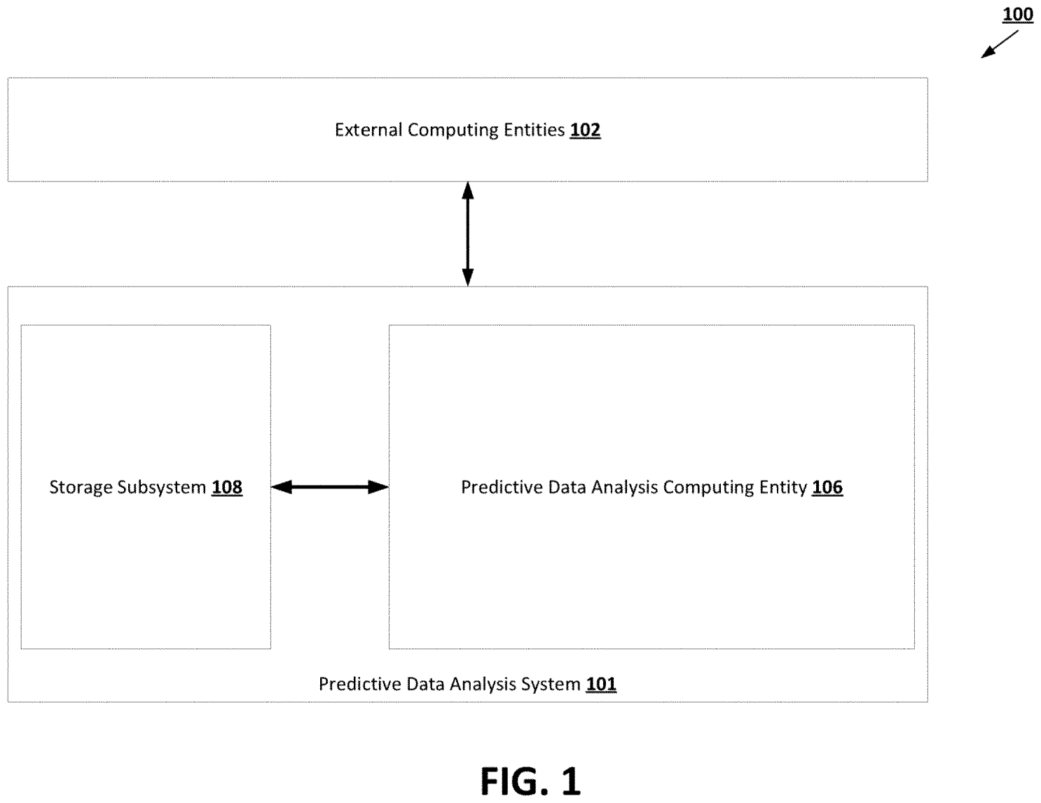

FIG. 1 is a schematic diagram of an example architecture 100 for performing predictive data analysis. The architecture 100 includes a predictive data analysis system 101 configured to receive predictive data analysis requests from external computing entities 102, process the predictive data analysis requests to generate predictions, provide the generated predictions to the external computing entities 102, and automatically perform prediction-based actions based at least in part on the generated predictions. An example of a prediction that can be generated using the predictive data analysis system 101 is a prediction about forecasted number of active cases, forecasted number of recovered cases, and forecasted number of deceased cases in relation to a disease spread scenario (e.g., a pandemic) based at least in part on historic timeseries data associated with the disease spread scenario.

In some embodiments, predictive data analysis system 101 may communicate with at least one of the external computing entities 102 using one or more communication networks. Examples of communication networks include any wired or wireless communication network including, for example, a wired or wireless local area network (LAN), personal area network (PAN), metropolitan area network (MAN), wide area network (WAN), or the like, as well as any hardware, software and/or firmware required to implement it (such as, e.g., network routers, and/or the like).

The predictive data analysis system 101 may include a predictive data analysis computing entity 106 and a storage subsystem 108. The predictive data analysis computing entity 106 may be configured to receive predictive data analysis requests from one or more external computing entities 102, process the predictive data analysis requests to generate predictions corresponding to the predictive data analysis requests, provide the generated predictions to the external computing entities 102, and automatically perform prediction-based actions based at least in part on the generated predictions.

The storage subsystem 108 may be configured to store input data used by the predictive data analysis computing entity 106 to perform predictive data analysis as well as model definition data used by the predictive data analysis computing entity 106 to perform various predictive data analysis tasks. The storage subsystem 108 may include one or more storage units, such as multiple distributed storage units that are connected through a computer network. Each storage unit in the storage subsystem 108 may store at least one of one or more data assets and/or one or more data about the computed properties of one or more data assets. Moreover, each storage unit in the storage subsystem 108 may include one or more non-volatile storage or memory media including, but not limited to, hard disks, ROM, PROM, EPROM, EEPROM, flash memory, MMCs, SD memory cards, Memory Sticks, CBRAM, PRAM, FeRAM, NVRAM, MRAM, RRAM, SONOS, FIG RAM, Millipede memory, racetrack memory, and/or the like.

### Exemplary Predictive Data Analysis Computing Entity

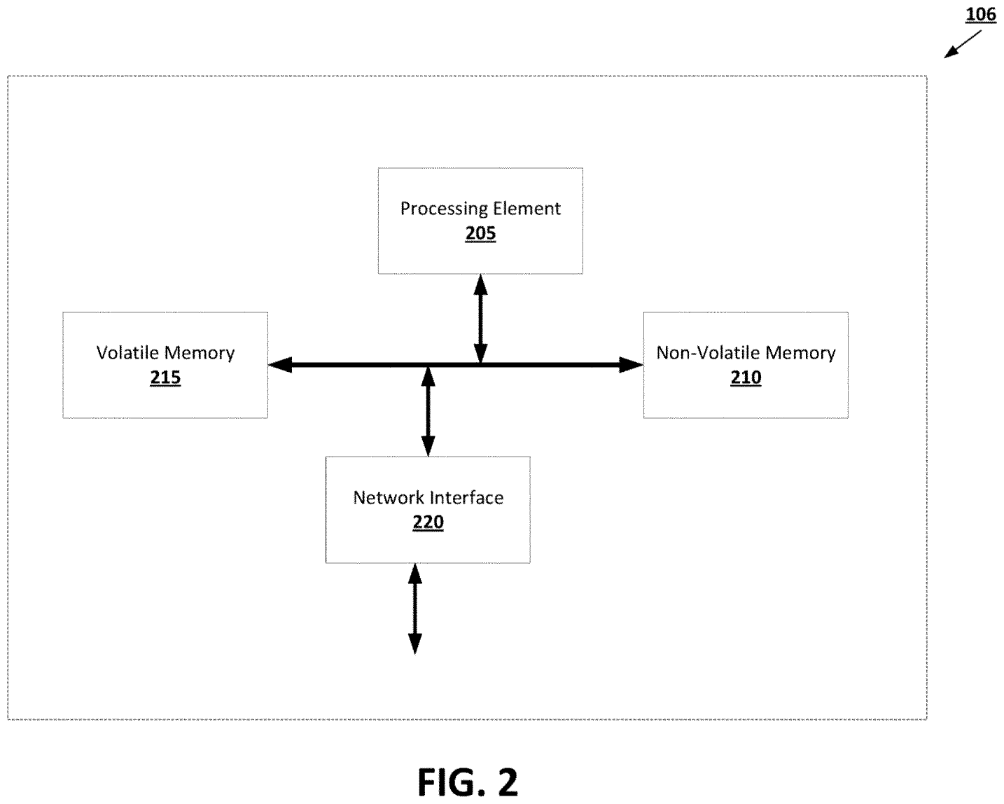

FIG. 2 provides a schematic of a predictive data analysis computing entity 106 according to one embodiment of the present invention. In general, the terms computing entity, computer, entity, device, system, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. Such functions, operations, and/or processes may include, for example, transmitting, receiving, operating on, processing, displaying, storing, determining, creating/generating, monitoring, evaluating, comparing, and/or similar terms used herein interchangeably. In one embodiment, these functions, operations, and/or processes can be performed on data, content, information, and/or similar terms used herein interchangeably.

As indicated, in one embodiment, the predictive data analysis computing entity 106 may also include one or more communications interfaces 220 for communicating with various computing entities, such as by communicating data, content, information, and/or similar terms used herein interchangeably that can be transmitted, received, operated on, processed, displayed, stored, and/or the like.

As shown in FIG. 2, in one embodiment, the predictive data analysis computing entity 106 may include, or be in communication with, one or more processing elements 205 (also referred to as processors, processing circuitry, and/or similar terms used herein interchangeably) that communicate with other elements within the predictive data analysis computing entity 106 via a bus, for example. As will be understood, the processing element 205 may be embodied in a number of different ways.

For example, the processing element 205 may be embodied as one or more complex programmable logic devices (CPLDs), microprocessors, multi-core processors, coprocessing entities, application-specific instruction-set processors (ASIPs), microcontrollers, and/or controllers. Further, the processing element 205 may be embodied as one or more other processing devices or circuitry. The term circuitry may refer to an entirely hardware embodiment or a combination of hardware and computer program products. Thus, the processing element 205 may be embodied as integrated circuits, application specific integrated circuits (ASICs), field programmable gate arrays (FPGAs), programmable logic arrays (PLAs), hardware accelerators, other circuitry, and/or the like.

As will therefore be understood, the processing element 205 may be configured for a particular use or configured to execute instructions stored in volatile or non-volatile media or otherwise accessible to the processing element 205. As such, whether configured by hardware or computer program products, or by a combination thereof, the processing element 205 may be capable of performing steps or operations according to embodiments of the present invention when configured accordingly.

In one embodiment, the predictive data analysis computing entity 106 may further include, or be in communication with, non-volatile media (also referred to as non-volatile storage, memory, memory storage, memory circuitry and/or similar terms used herein interchangeably). In one embodiment, the non-volatile storage or memory may include one or more non-volatile storage or memory media 210, including, but not limited to, hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

As will be recognized, the non-volatile storage or memory media may store databases, database instances, database management systems, data, applications, programs, program modules, scripts, source code, object code, byte code, compiled code, interpreted code, machine code, executable instructions, and/or the like. The term database, database instance, database management system, and/or similar terms used herein interchangeably may refer to a collection of records or data that is stored in a computer-readable storage medium using one or more database models, such as a hierarchical database model, network model, relational model, entity-relationship model, object model, document model, semantic model, graph model, and/or the like.

In one embodiment, the predictive data analysis computing entity 106 may further include, or be in communication with, volatile media (also referred to as volatile storage, memory, memory storage, memory circuitry and/or similar terms used herein interchangeably). In one embodiment, the volatile storage or memory may also include one or more volatile storage or memory media 215, including, but not limited to, RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like.

As will be recognized, the volatile storage or memory media may be used to store at least portions of the databases, database instances, database management systems, data, applications, programs, program modules, scripts, source code, object code, byte code, compiled code, interpreted code, machine code, executable instructions, and/or the like being executed by, for example, the processing element 205. Thus, the databases, database instances, database management systems, data, applications, programs, program modules, scripts, source code, object code, byte code, compiled code, interpreted code, machine code, executable instructions, and/or the like may be used to control certain aspects of the operation of the predictive data analysis computing entity 106 with the assistance of the processing element 205 and operating system.

As indicated, in one embodiment, the predictive data analysis computing entity 106 may also include one or more communications interfaces 220 for communicating with various computing entities, such as by communicating data, content, information, and/or similar terms used herein interchangeably that can be transmitted, received, operated on, processed, displayed, stored, and/or the like. Such communication may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. Similarly, the predictive data analysis computing entity 106 may be configured to communicate via wireless external communication networks using any of a variety of protocols, such as general packet radio service (GPRS), Universal Mobile Telecommunications System (UMTS), Code Division Multiple Access 2000 (CDMA2000), CDMA2000 1X (1×RTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.11 (Wi-Fi), Wi-Fi Direct, 802.16 (WiMAX), ultra-wideband (UWB), infrared (IR) protocols, near field communication (NFC) protocols, Wibree, Bluetooth protocols, wireless universal serial bus (USB) protocols, and/or any other wireless protocol.

Although not shown, the predictive data analysis computing entity 106 may include, or be in communication with, one or more input elements, such as a keyboard input, a mouse input, a touch screen/display input, motion input, movement input, audio input, pointing device input, joystick input, keypad input, and/or the like. The predictive data analysis computing entity 106 may also include, or be in communication with, one or more output elements (not shown), such as audio output, video output, screen/display output, motion output, movement output, and/or the like.

### Exemplary External Computing Entity

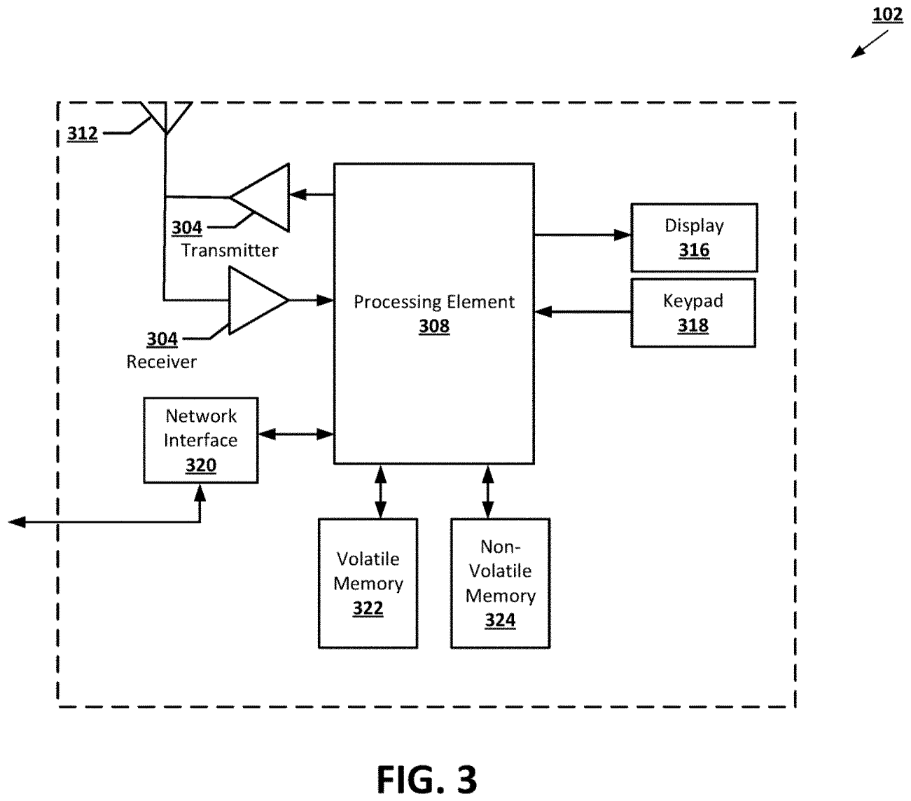

FIG. 3 provides an illustrative schematic representative of an external computing entity 102 that can be used in conjunction with embodiments of the present invention. In general, the terms device, system, computing entity, entity, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. External computing entities 102 can be operated by various parties. As shown in FIG. 3, the external computing entity 102 can include an antenna 312, a transmitter 304 (e.g., radio), a receiver 306 (e.g., radio), and a processing element 308 (e.g., CPLDs, microprocessors, multi-core processors, coprocessing entities, ASIPs, microcontrollers, and/or controllers) that provides signals to and receives signals from the transmitter 304 and receiver 306, correspondingly.

The signals provided to and received from the transmitter 304 and the receiver 306, correspondingly, may include signaling information/data in accordance with air interface standards of applicable wireless systems. In this regard, the external computing entity 102 may be capable of operating with one or more air interface standards, communication protocols, modulation types, and access types. More particularly, the external computing entity 102 may operate in accordance with any of a number of wireless communication standards and protocols, such as those described above with regard to the predictive data analysis computing entity 106. In a particular embodiment, the external computing entity 102 may operate in accordance with multiple wireless communication standards and protocols, such as UMTS, CDMA2000, 1×RTT, WCDMA, GSM, EDGE, TD-SCDMA, LTE, E-UTRAN, EVDO, HSPA, HSDPA, Wi-Fi, Wi-Fi Direct, WiMAX, UWB, IR, NFC, Bluetooth, USB, and/or the like. Similarly, the external computing entity 102 may operate in accordance with multiple wired communication standards and protocols, such as those described above with regard to the predictive data analysis computing entity 106 via a network interface 320.

Via these communication standards and protocols, the external computing entity 102 can communicate with various other entities using concepts such as Unstructured Supplementary Service Data (USSD), Short Message Service (SMS), Multimedia Messaging Service (MIMS), Dual-Tone Multi-Frequency Signaling (DTMF), and/or Subscriber Identity Module Dialer (SIM dialer). The external computing entity 102 can also download changes, add-ons, and updates, for instance, to its firmware, software (e.g., including executable instructions, applications, program modules), and operating system.

According to one embodiment, the external computing entity 102 may include location determining aspects, devices, modules, functionalities, and/or similar words used herein interchangeably. For example, the external computing entity 102 may include outdoor positioning aspects, such as a location module adapted to acquire, for example, latitude, longitude, altitude, geocode, course, direction, heading, speed, universal time (UTC), date, and/or various other information/data. In one embodiment, the location module can acquire data, sometimes known as ephemeris data, by identifying the number of satellites in view and the relative positions of those satellites (e.g., using global positioning systems (GPS)). The satellites may be a variety of different satellites, including Low Earth Orbit (LEO) satellite systems, Department of Defense (DOD) satellite systems, the European Union Galileo positioning systems, the Chinese Compass navigation systems, Indian Regional Navigational satellite systems, and/or the like. This data can be collected using a variety of coordinate systems, such as the Decimal Degrees (DD); Degrees, Minutes, Seconds (DMS); Universal Transverse Mercator (UTM); Universal Polar Stereographic (UPS) coordinate systems; and/or the like. Alternatively, the location information/data can be determined by triangulating the external computing entity's 102 position in connection with a variety of other systems, including cellular towers, Wi-Fi access points, and/or the like. Similarly, the external computing entity 102 may include indoor positioning aspects, such as a location module adapted to acquire, for example, latitude, longitude, altitude, geocode, course, direction, heading, speed, time, date, and/or various other information/data. Some of the indoor systems may use various position or location technologies including RFID tags, indoor beacons or transmitters, Wi-Fi access points, cellular towers, nearby computing devices (e.g., smartphones, laptops) and/or the like. For instance, such technologies may include the iBeacons, Gimbal proximity beacons, Bluetooth Low Energy (BLE) transmitters, NFC transmitters, and/or the like. These indoor positioning aspects can be used in a variety of settings to determine the location of someone or something to within inches or centimeters.

The external computing entity 102 may also comprise a user interface (that can include a display 316 coupled to a processing element 308) and/or a user input interface (coupled to a processing element 308). For example, the user interface may be a user application, browser, user interface, and/or similar words used herein interchangeably executing on and/or accessible via the external computing entity 102 to interact with and/or cause display of information/data from the predictive data analysis computing entity 106, as described herein. The user input interface can comprise any of a number of devices or interfaces allowing the external computing entity 102 to receive data, such as a keypad 318 (hard or soft), a touch display, voice/speech or motion interfaces, or other input device. In embodiments including a keypad 318, the keypad 318 can include (or cause display of) the conventional numeric (0-9) and related keys (#, *), and other keys used for operating the external computing entity 102 and may include a full set of alphabetic keys or set of keys that may be activated to provide a full set of alphanumeric keys. In addition to providing input, the user input interface can be used, for example, to activate or deactivate certain functions, such as screen savers and/or sleep modes.

The external computing entity 102 can also include volatile storage or memory 322 and/or non-volatile storage or memory 324, which can be embedded and/or may be removable. For example, the non-volatile memory may be ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like. The volatile memory may be RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like. The volatile and non-volatile storage or memory can store databases, database instances, database management systems, data, applications, programs, program modules, scripts, source code, object code, byte code, compiled code, interpreted code, machine code, executable instructions, and/or the like to implement the functions of the external computing entity 102. As indicated, this may include a user application that is resident on the entity or accessible through a browser or other user interface for communicating with the predictive data analysis computing entity 106 and/or various other computing entities.

In another embodiment, the external computing entity 102 may include one or more components or functionality that are the same or similar to those of the predictive data analysis computing entity 106, as described in greater detail above. As will be recognized, these architectures and descriptions are provided for exemplary purposes only and are not limiting to the various embodiments.

In various embodiments, the external computing entity 102 may be embodied as an artificial intelligence (AI) computing entity, such as an Amazon Echo, Amazon Echo Dot, Amazon Show, Google Home, and/or the like. Accordingly, the external computing entity 102 may be configured to provide and/or receive information/data from a user via an input/output mechanism, such as a display, a camera, a speaker, a voice-activated input, and/or the like. In certain embodiments, an AI computing entity may comprise one or more predefined and executable program algorithms stored within an onboard memory storage module, and/or accessible over a network. In various embodiments, the AI computing entity may be configured to retrieve and/or execute one or more of the predefined program algorithms upon the occurrence of a predefined trigger event.

### V. Exemplary System Operations

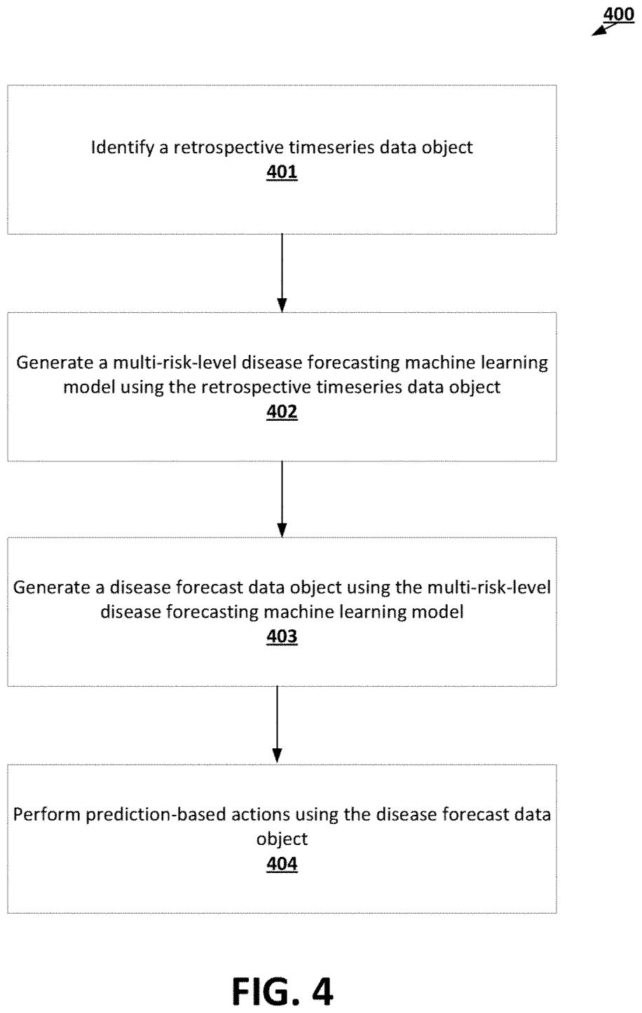

FIG. 4 is a flowchart diagram of an example process 400 for performing optimization-based disease spread forecasting using a multi-risk-level disease spread forecasting machine learning model. Via the various steps/operations of the process 400, a predictive data analysis computing entity 106 can generate reliable disease spread forecasts (e.g., pandemic spread forecasts) by using machine learning techniques that: (i) integrate predictive insights about varying levels of risk among susceptible population, (ii) integrate predictive insights about varying exposure/transition probabilities across time by using temporally dynamic parameters, and/or (iii) utilize a retrospective timeseries data object to calculate error measures that can in turn be used to determine inferred susceptibility parameters rather than constant susceptibility parameters.

The process 400 begins at step/operation 401 when the predictive data analysis computing entity 106 identifies a retrospective timeseries data object. The retrospective timeseries data object may describe one or more observed metrics related to disease spread across one or more time units. For example, the retrospective timeseries data object may describe observed metrics about at least one of currently infected cases (aka. active cases) of a disease across one or more time units (e.g., one or more days), recovered cases of a disease across one or more time units, and deceased cases (aka. terminated cases) of a disease across one or more time units. In some embodiments, the retrospective timeseries data object describe one or more observed metrics about disease spread for a target period of time, such as a latest recorded time unit (i.e., a latest recorded time unit, such as a current day). Examples of such observed metrics may include one or more of retrospective susceptibility count (Sₜ) values, retrospective containment count (Qₜ) values, retrospective recovery count (Rₜ) values, retrospective termination count (Dₜ) values, retrospective infection count (Iₜ) values, retrospective heightened risk count values (Eₜ), retrospective non-heightened risk count values (Nₜ), and a total population count (N).

A retrospective susceptibility count, which is also referred to herein as Sₜ, may describe a number of individuals deemed to be susceptible to a particular disease during a target time period t, such as during a current time period (e.g., during a current day). As described above, in some embodiments, Sₜ may be described by a retrospective timeseries data object. In some other embodiments, Sₜ may be estimated based at least in part on a total population count (N) at the target period, a general susceptibility parameter (a or alpha), a retrospective recovery count (Rₜ), and a recovered susceptibility parameter (a′ or alpha′).

A retrospective heightened risk count, which is also referred to herein as Eₜ, may describe a number of individuals deemed to be having a higher level of risk of disease spread relative to the normal population during a target time period t, such as during a current time period (e.g., during a current day). This may include essential workers, healthcare workers, people with physiological conditions that are deemed to be associated with heightened risk of disease spread, and/or the like. As described above, in some embodiments, Eₜ may be described by a retrospective timeseries data object. In some other embodiments, Eₜ may be estimated based at least in part on a retrospective susceptibility count (Sₜ) and a heightened risk ratio parameter (λ or lambda).

A retrospective non-heightened risk count, which is also referred to herein as Nₜ, may describe a number of individuals deemed to be having a risk of disease spread that is equivalent to the risk of disease spread of the normal population during a target time period t, such as during a current time period (e.g., during a current day). This may include grocery store shoppers and/or retail shoppers. As described above, in some embodiments, Nₜ may be described by a retrospective timeseries data object. In some other embodiments, Nₜ may be estimated based at least in part on a retrospective susceptibility count (Sₜ) and a non-heightened risk ratio parameter (1-λ or 1-lambda).

A retrospective infection count, which is also referred to herein as Iₜ, may describe a number of individuals deemed to be currently infected by a disease during a target time period t, such as during a current time period (e.g., during a current day). As described above, in some embodiments, Iₜ may be described by a retrospective timeseries data object, and may for example be determined based at least in part on the number of active cases for the target time period as described by the retrospective timeseries data object. In some other embodiments, Iₜ may be estimated based at least in part on a retrospective heightened risk count (Eₜ), a heightened risk ifection probability parameter (γ or gamma), a retrospective non-heightened risk count (Iₜ), and a non-heightened risk infection probability parameter (β or Beta).

A retrospective containment count, which is also referred to herein as Qₜ, may describe a number of individuals that are deemed infected but are also deemed to be at a minimal risk of disease spread due to physical containment of those individuals during a target time period t, such as during a current time period (e.g., during a current day). This may include quarantined individuals, individuals under lockdown measures, individuals under stay-at-home measures, individuals under curfew measures, and/or the like. As described above, in some embodiments, Qₜ may be described by a retrospective timeseries data object. In some other embodiments, Qₜ may be estimated based at least in part on a retrospective infection count (Iₜ) and a containment probability parameter (Θ or theta).

A retrospective termination count, which is also referred to herein as Dₜ, may describe a number of individuals deemed to be eliminated from the total population as a result of the disease. This may include deceased individuals, brain-dead individuals, and/or the like. As described above, in some embodiments, Dₜ may be described by a retrospective timeseries data object. In some other embodiments, Dₜ may be estimated based at least in part on a retrospective containment count (Qₜ) and a termination probability parameter (ρ or rho).

A retrospective recovery count, which is also referred to herein as Rₜ, may describe a number of individuals deemed to have been infected with the disease and subsequently recovered from the disease. As described above, in some embodiments, Rₜ may be described by a retrospective timeseries data object. In some other embodiments, Rₜ may be estimated based at least in part on a retrospective containment count (Qₜ) and a recovery probability parameter (δ or delta).

At step/operation 402, the predictive data analysis computing entity 106 generates the multi-risk-level disease spread forecasting machine learning model based at least in part on the retrospective timeseries data object. The multi-level disease spread forecasting machine learning model may be a machine learning model (e.g., a statistical model with one or more trained parameters, a model using a system of differential equations with one or more configurable parameters, and/or the like) that generates a disease spread forecast using predictive insights that relate to differing exposure to disease spread among at least two categories of the susceptible population. As one example, a multi-level disease spread forecasting machine learning model may integrate predictive insights about differing exposure to disease spread among a heightened risk portion of the susceptible population (e.g., an essential worker segment of the susceptible population) and a non-heightened risk portion of the susceptible population (e.g., a retail shopper segment of the susceptible population). As another example, a multi-level disease spread forecasting machine learning model may integrate predictive insights about differing exposure to disease spread among a heightened risk portion of the susceptible population (e.g., an essential worker segment of the susceptible population), a non-heightened risk portion of the susceptible population (e.g., a retail shopper segment of the susceptible population), and a minimal-risk portion of the susceptible population (e.g., a quarantined segment of the susceptible population). As yet another example, a multi-level disease spread forecasting machine learning model may integrate predictive insights about differing exposure to disease spread among a heightened risk portion of the susceptible population (e.g., a segment of the susceptible population that includes essential workers working in contained spaces), a non-heightened risk portion of the susceptible population (e.g., a retail shopper segment of the susceptible population), and a medium risk portion of the susceptible population (e.g., a segment of the susceptible population that includes essential workers working in open spaces). As a further example, a multi-level disease spread forecasting machine learning model may integrate predictive insights about differing exposure to disease spread among a heightened risk portion of the susceptible population (e.g., a segment of the susceptible population that includes essential workers working in contained spaces), a non-heightened risk portion of the susceptible population (e.g., a retail shopper segment of the susceptible population), a medium risk portion of the susceptible population (e.g., a segment of the susceptible population that includes essential workers working in open spaces), and a minimal-risk portion of the susceptible population (e.g., a quarantined segment of the susceptible population).

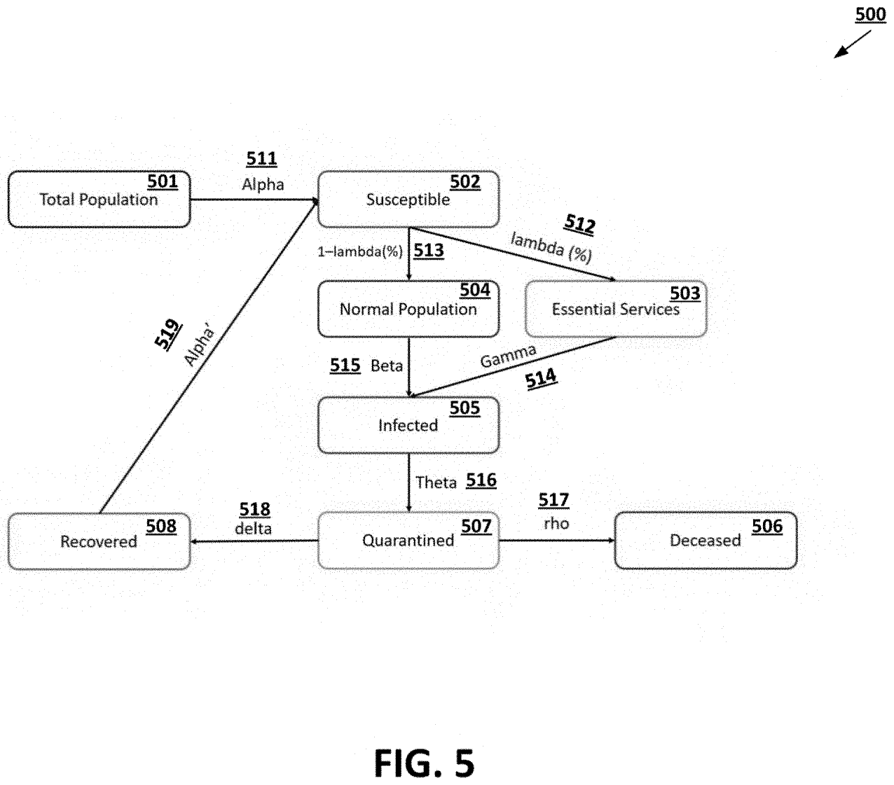

An operational example of operations of a multi-risk-level disease spread forecasting machine learning model 500 is depicted in FIG. 5. As depicted in FIG. 5, the multi-risk-level disease spread forecasting machine learning model 500 models a retrospective susceptibility count 502 (Sₜ) as a sum of two values: (i) the output of applying a general susceptibility parameter 511 (α or alpha) to the retrospective total population 501 (N), and (ii) the output of applying a recovered susceptibility parameter 519 (α′ or alpha′) to a retrospective recovery count 508 (Rₜ). The general susceptibility parameter and the recovered susceptibility parameter 519 are described in greater detail below.

The general susceptibility parameter, which is also referred to herein as a or alpha, may describe an estimated likelihood that a member of a population may be susceptible to disease spread, e.g., an expected/estimated/measured ratio of a total population that is likely to be susceptible to disease spread. In some embodiments, a is a temporally dynamic parameter, i.e., α is a parameter that is updated based at least in part on newly arriving data. For example, in some embodiments, α may be determined in accordance with an optimization technique configured to minimize a measure of error between disease spread forecasts across historical data and ground-truth disease spread metrics determined using the historical data.

The recovered susceptibility parameter, which is also referred to herein as α′ or alpha′, may describe an estimated likelihood that a member of a recovered population may be susceptible to disease spread, e.g., an expected/estimated/measured ratio of the recovered population that is likely to still be susceptible to disease spread. In some embodiments, α′ is determined based at least in part on scientific literature and/or analytical studies. In some embodiments, α′ is expected to be lower than α. In some embodiments, α′may be determined in accordance with an optimization technique configured to minimize a measure of error between disease spread forecasts across historical data and ground-truth disease spread metrics determined using the historical data. In some embodiments, α′ is a temporally dynamic parameter, i.e., α′ is a parameter that is updated based at least in part on newly arriving data.

As further depicted in FIG. 5, the multi-risk-level disease spread forecasting machine learning model 500 models a retrospective heightened risk count 503 (Eₜ) as the output of applying a heightened risk growth parameter 512 (X, or lambda) to the retrospective susceptibility count 502 (Sₜ). The heightened risk growth parameter, which is also referred to herein as λ or lambda, may describe an estimated likelihood that a member of a susceptible population may be part of a heightened risk segment of the susceptible population, such as an essential worker segment of the susceptible population. For example, λ may describe a heightened risk ratio of a susceptible population. In some embodiments, λ is determined based at least in part on preexisting data, such as economic data. In some embodiments, X, may be determined in accordance with an optimization technique configured to minimize a measure of error between disease spread forecasts across historical data and ground-truth disease spread metrics determined using the historical data. In some embodiments, λ is a temporally dynamic parameter, i.e., λ is a parameter that is updated based at least in part on newly data.

As further depicted in FIG. 5, the multi-risk-level disease spread forecasting machine learning model 500 models a retrospective non-heightened risk count 504 (Nₜ) as the output of applying a non-heightened risk growth parameter 513 (1-λ or 1-lambda) to the retrospective susceptibility count 502 (Sₜ). The non-heightened risk growth parameter, which is also referred to herein as 1-λ or 1-lambda, may describe an estimated likelihood that a member of a susceptible population may be part of a non-heightened risk segment of the susceptible population, such as a regular retail worker segment of the susceptible population. For example, 1-λ may describe a non-heightened risk ratio of a susceptible population. In some embodiments, 1-λ is determined based at least in part on preexisting data, such as economic data. In some embodiments, 1-λ may be determined in accordance with an optimization technique configured to minimize a measure of error between disease spread forecasts across historical data and ground-truth disease spread metrics determined using the historical data. In some embodiments, 1-λ is a temporally dynamic parameter, i.e., 1-λ is a parameter that is updated based at least in part on newly data and/or based at least in part on updates to λ across time.

As further depicted in FIG. 5, the multi-risk-level disease spread forecasting machine learning model 500 models a retrospective infection count 505 (Iₜ) as the output of summing two values: (i) the output of applying a heightened risk infection probability parameter 514 (γ or gamma) to the retrospective heightened risk count 503 (Eₜ), and (ii) the output of applying a non-heightened risk infection probability parameter 515 (β or beta) to the retrospective non-heightened risk count 504 (Nₜ). The heightened risk infection probability parameter 514 (γ or gamma) and the non-heightened risk infection probability parameter 515 (β or beta) are described in greater detail below.

The heightened risk infection probability parameter, which is also referred to herein as γ or gamma, may describe an estimated likelihood that a member of a heightened risk population may be infected to a disease. For example, γ may describe a historical ratio of heightened risk individuals (e.g., essential works) who have been infected with the disease. In some embodiments, γ may be determined in accordance with an optimization technique configured to minimize a measure of error between disease spread forecasts across historical data and ground-truth disease spread metrics determined using the historical data. In some embodiments, γ is a temporally dynamic parameter, i.e., γ is a parameter that is updated based at least in part on newly data.

The non-heightened risk infection probability parameter, which is also referred to herein as β or beta, may describe an estimated likelihood that a member of a non-heightened risk population may be infected to a disease. For example, β may describe a historical ratio of non-heightened risk individuals (e.g., retail shoppers) who have been infected with the disease. In some embodiments, β may be determined in accordance with an optimization technique configured to minimize a measure of error between disease spread forecasts across historical data and ground-truth disease spread metrics determined using the historical data. In some embodiments, β is a temporally dynamic parameter, i.e., β is a parameter that is updated based at least in part on newly data.

As further depicted in FIG. 5, the multi-risk-level disease spread forecasting machine learning model 500 models a retrospective containment count (Qₜ) 507 as the output of applying a containment probability parameter 516 (Θ or theta) to the retrospective infection count 505 (Iₜ). The containment probability parameter, which is also referred to herein as Θ or theta, may describe an estimated likelihood that a member of the infected population may be contained, e.g., a ratio of the infected individuals that are estimated to have been quarantined. In some embodiments, Θ is determined based at least in part on predefined data, e.g., social studies data. In some embodiments, Θ may be determined in accordance with an optimization technique configured to minimize a measure of error between disease spread forecasts across historical data and ground-truth disease spread metrics determined using the historical data. In some embodiments, Θ is a temporally dynamic parameter, i.e., Θ is a parameter that is updated based at least in part on newly data.

As further depicted in FIG. 5, the multi-risk-level disease spread forecasting machine learning model 500 models a retrospective termination count (Dₜ) 506 as the output of applying a termination probability parameter 517 (ρ or rho) to the retrospective containment count 507 (Qₜ). The termination probability parameter, which is also referred to herein as ρ or rho, may describe an estimated likelihood that a member of the quarantined population may decease/terminate, e.g., a ratio of the quarantined individuals that are estimated to have been deceased. In some embodiments, ρ is determined based at least in part on predefined data, e.g., death statistics data. In some embodiments, ρ may be determined in accordance with an optimization technique configured to minimize a measure of error between disease spread forecasts across historical data and ground-truth disease spread metrics determined using the historical data. In some embodiments, ρ is a temporally dynamic parameter, i.e., ρ is a parameter that is updated based at least in part on newly data.

As further depicted in FIG. 5, the multi-risk-level disease spread forecasting machine learning model 500 models a retrospective recovery count (Rₜ) 508 as the output of applying a recovery probability parameter 517 (δ or delta) to the retrospective containment count 507 (Qₜ). The recovery probability parameter, which is also referred to herein as δ or delta, may describe an estimated likelihood that a member of the quarantined population may recover, e.g., a ratio of the quarantined individuals that are estimated to have been recovered. In some embodiments, δ is determined based at least in part on predefined data, e.g., recovery statistics data, hospitalization data, and/or the like. In some embodiments, δ may be determined in accordance with an optimization technique configured to minimize a measure of error between disease spread forecasts across historical data and ground-truth disease spread metrics determined using the historical data. In some embodiments, δ is a temporally dynamic parameter, i.e., δ is a parameter that is updated based at least in part on newly data.

In some embodiments, the multi-risk-level disease spread forecasting machine learning model includes a group of sub-models that may be solved as a group of differential equation sub-models. For example, the multi-risk-level disease spread forecasting machine learning model may include a prospective susceptibility forecast determination sub-model that is configured to determine a prospective susceptibility forecast

$$( \frac{dS ( t )}{dt} )$$

based at least in part on: (i) the retrospective infection count value (Iₜ), (ii) the retrospective susceptibility count value (Sₜ), (iii) the heightened risk ratio parameter (λ), (iv) the heightened risk infection probability parameter (γ), (v) the non-heightened risk infection probability parameter (β), and (vi) the general susceptibility parameter (α). In some embodiments, the prospective susceptibility forecast determination sub-model may perform operations of the below equation:

$$\frac{dS ( t )}{dt} = [ ( 1 - λ ) * β + λ * γ ] * {I}_{t} * \frac{{S}_{t}}{α * N} \qquad Equation 1$$

As another example, the multi-risk-level disease spread forecasting machine learning model may include a prospective heightened risk forecast determination sub-model that is configured to determine a prospective heightened risk forecast

$$( \frac{dE ( t )}{dt} )$$

based at least in part on: the prospective susceptibility forecast

$$( \frac{dS ( t )}{dt} ) ,$$

the retrospective heightened risk count value (Eₜ), and the heightened risk infection probability parameter (γ). In some embodiments, the prospective heightened risk forecast determination sub-model may perform operations of the below equation:

$$\frac{dE ( t )}{dt} = \frac{dS ( t )}{dt} - γ * {E}_{t} \qquad Equation 2$$

As yet another example, the multi-risk-level disease spread forecasting machine learning model may include a prospective non-heightened risk forecast determination sub-model that is configured to determine a prospective non-heightened risk forecast

$$( \frac{dN ( t )}{dt} )$$

based at least in part on: the prospective susceptibility forecast

$$( \frac{dS ( t )}{dt} ) ,$$

the retrospective non-heightened risk count value (Nₜ), and the non-heightened risk infection probability parameter (β). In some embodiments, the prospective non-heightened risk forecast determination sub-model may perform operations of the below equation:

$$\frac{dN ( t )}{dt} = \frac{dS ( t )}{dt} - β * {N}_{t} \qquad Equation 3$$

As an additional example, the multi-risk-level disease spread forecasting machine learning model may include a prospective infection forecast determination sub-model that is configured to determine a prospective infection forecast

$$( \frac{dI ( t )}{dt} )$$

based at least in part on: the prospective susceptibility forecast

$$( \frac{dS ( t )}{dt} ) ,$$

the retrospective infection count value (Iₜ), and the containment probability parameter (Θ). In some embodiments, the prospective infection forecast determination sub-model may perform operations of the below equation:

$$\frac{dI ( t )}{dt} = \frac{dS ( t )}{dt} - Θ * {I}_{t} \qquad Equation 4$$

As yet another example, the multi-risk-level disease spread forecasting machine learning model may include a prospective containment forecast determination sub-model that is configured to determine a prospective containment forecast

$$( \frac{dQ ( t )}{dt} )$$

based at least in part on: (i) the respective infection count (Iₜ), (ii) the respective containment count (Qₜ), (iii) the containment probability parameter (Θ), (iv) the recovery probability parameter (δ), and (v) the termination probability parameter (ρ). In some embodiments, the prospective containment forecast determination sub-model may perform operations of the below equation:

$$\frac{dQ ( t )}{dt} = Θ * {I}_{t} - δ * {Q}_{t} - ρ * {Q}_{t} \qquad Equation 5$$

As yet another example, the multi-risk-level disease spread forecasting machine learning model may include a prospective recovery forecast determination sub-model that is configured to determine a prospective recovery forecast

$$( \frac{dR ( t )}{dt} )$$

based at least in part on the respective containment count (Qₜ) and the recovery probability parameter (δ). In some embodiments, prospective recovery forecast determination sub-model may perform operations of the below equation:

$$\frac{dR ( t )}{dt} = δ * {Q}_{t} \qquad Equation 6$$

As a further example, the multi-risk-level disease spread forecasting machine learning model may include a prospective termination forecast determination sub-model that is configured to determine a prospective recovery forecast

$$( \frac{dD ( t )}{dt} )$$

based at least in part on the respective containment count (Qₜ) and the termination probability parameter (ρ). In some embodiments, prospective termination forecast determination sub-model may perform operations of the below equation:

$$\frac{dD ( t )}{dt} = ρ * {Q}_{t} \qquad Equation 7$$

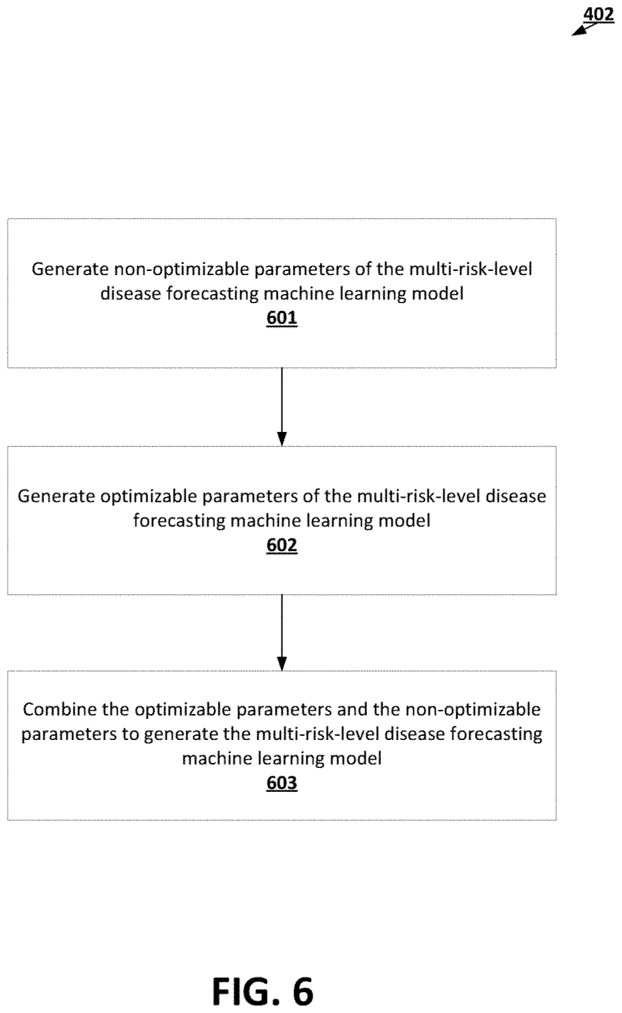

In some embodiments, step/operation 402 may be performed in accordance with the process depicted in FIG. 6. The process depicted in FIG. 6 begins at step/operation 601 when the predictive data analysis computing entity 106 determines non-optimizable parameters of the multi-risk-level disease spread forecasting machine learning model. The non-optimizable parameters of the multi-risk-level disease spread forecasting machine learning model are those parameters that are determined based at least in part on observational data and/or based at least in part on ground-truth data. For example, in some embodiments, λ is determined based at least in part on preexisting data, such as economic data describing a portion of the population working in essential services jobs. As another example, in some embodiments, γ may be determined based at least in part on a historical ratio of heightened risk individuals (e.g., essential works) who have been infected with the disease and/or may be determined based at least in part on scientific data. As yet another example, in some embodiments, β may be determined based at least in part on a historical ratio of non-heightened risk individuals (e.g., retail shoppers) who have been infected with the disease and/or may be determined based at least in part on scientific data. As a further example, in some embodiments, may be is determined based at least in part on social studies data. As yet another example, in some embodiments, ρ may be determined based at least in part on death statistics data.

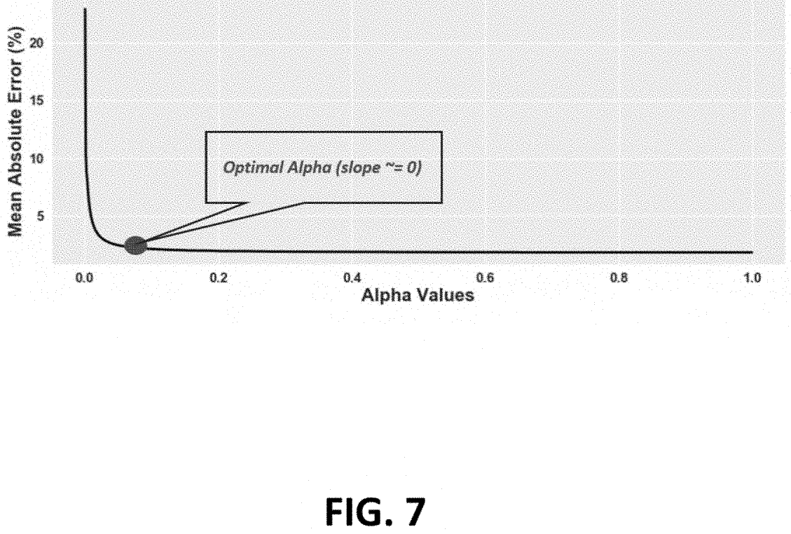

At step/operation 602, the predictive data analysis computing entity 106 determines optimizable parameters of the multi-risk-level disease spread forecasting machine learning model. Examples of optimizable parameters may include the susceptibility parameter (α). In some embodiments, α is determined in a manner that minimizes a measure of error of forecasts performed using an optimum value of α. For example, in some embodiments, the predictive data analysis computing entity 106 may fix the values of the non-optimizable parameters of the multi-risk-level disease spread forecasting machine learning model, then generate a mean squared error (MAE) measure for each candidate value of a in order to generate one or more target forecasts, and then compare the target forecasts to ground-truth data provided by historical data in order to determine an optimal value of α, such as the least value of a that causes the MAE graph to have a slope of zero or near-zero (e.g., within a 0.001 range of zero). An operational example of determining an optimal value for α using the noted optimization technique is depicted in FIG. 7, which selects a value of α configured to cause the MAE graph to have a near-zero slope.

At step/operation 603, the predictive data analysis computing entity 106 combines the optimizable parameters and the non-optimizable parameters to generate the trained multi-risk-level disease spread forecasting machine learning model. In some embodiments, subsequent to generating the trained multi-risk-level disease spread forecasting machine learning model, the predictive data analysis computing entity 106 enables access (e.g., to an external computing entity 102, such as a client computing entity) to the trained multi-risk-level disease spread forecasting machine learning model, which may enable using the trained multi-risk-level disease spread forecasting machine learning model to generate disease spread forecasts and to perform prediction-based actions based at least in part on the generated disease spread forecasts. In some embodiments, the predictive data analysis computing entity 106 may utilize the trained multi-risk-level disease spread forecasting machine learning model to generate disease forecasts and perform prediction-based actions, for example using the techniques described below in relation to step/operation 403 and step/operation 404.

Returning to FIG. 4, at step/operation 403, the predictive data analysis computing entity 106 generates a disease spread forecast data object using the multi-risk-level disease spread forecasting machine learning model. In some embodiments, the disease spread forecast data object may describe at least one or more forecasted/predicted metrics associated with spread of a disease during one or more prospective time periods. For example, the disease spread forecast data object may describe forecasted/predicted metrics associated with spread of a disease during a subsequent day/week/month. Examples of data entries that may be described by a disease spread forecast data object include a prospective susceptibility forecast

$$( \frac{dS ( t )}{dt} ) ,$$

a prospective heightened risk forecast

$$( \frac{dE ( t )}{dt} ) ,$$

a prospective non-heightened risk forecast

$$( \frac{dN ( t )}{dt} ) ,$$

a prospective infection forecast

$$( \frac{dI ( t )}{dt} ) ,$$

a prospective containment forecast

$$( \frac{dQ ( t )}{dt} )$$

a prospective recovery forecast

$$( \frac{dR ( t )}{dt} )$$

and a prospective termination forecast

$$( \frac{dD ( t )}{dt} ) .$$

A prospective susceptibility forecast, which is also referred to herein as

$$( \frac{dS ( t )}{dt} ) ,$$

may describe a predicted change in the quantity of a susceptible population between a current time period and a future time period. In some embodiments, the prospective susceptibility forecast may be determined based at least in part on: (i) the retrospective infection count value (Iₜ), (ii) the retrospective susceptibility count value (Sₜ), (iii) the heightened risk ratio parameter (λ), (iv) the heightened risk infection probability parameter (γ), (v) the non-heightened risk infection probability parameter (β), and (vi) the general susceptibility parameter (α).

A prospective heightened risk forecast, which is also referred to herein as

$$( \frac{dE ( t )}{dt} ) ,$$

may describe a predicted change in the quantity of a heightened risk population (e.g., an essential worker population) between a current time period and a future time period. In some embodiments, the prospective heightened risk forecast may be determined based at least in part on: the prospective susceptibility forecast

$$( \frac{dS ( t )}{dt} ) ,$$

the retrospective heightened risk count value (Eₜ), and the heightened risk infection probability parameter (γ).

A prospective non-heightened risk forecast, which is also referred to herein as

$$( \frac{dN ( t )}{dt} ) ,$$

may describe a predicted change in the quantity of a non-heightened risk population (e.g., a retail shopper population) between a current time period and a future time period. In some embodiments, the prospective non-heightened risk forecast may be determined based at least in part on: the prospective susceptibility forecast

$$( \frac{dS ( t )}{dt} ) ,$$

the retrospective non-heightened risk count value (Nₜ), and the non-heightened risk infection probability parameter (β).

A prospective infection forecast, which is also referred to herein as

$$\frac{dI ( t )}{dt} ,$$

may describe a predicted change in the quantity of an infected population between a current time period and a future time period. In some embodiments, the prospective infection forecast may be determined based at least in part on: the prospective susceptibility forecast

$$( \frac{dS ( t )}{dt} ) ,$$

the retrospective infection count value (Iₜ), and the containment probability parameter (Θ).

A prospective containment forecast, which is also referred to herein as

$$\frac{dQ ( t )}{dt} ,$$

may describe a predicted change in the quantity of an infected but contained (e.g., quarantined) population between a current time period and a future time period. In some embodiments, the prospective contained forecast may be determined based at least in part on: (i) the respective infection count (Iₜ), (ii) the respective containment count (Qₜ), (iii) the containment probability parameter (Θ), (iv) the recovery probability parameter (δ), and (v) the termination probability parameter (ρ).

A prospective recovery forecast, which is also referred to herein as

$$\frac{dR ( t )}{dt} ,$$

may describe a predicted change in the quantity of an infected but recovered population between a current time period and a future time period. In some embodiments, the prospective recovery forecast may be determined based at least in part on the respective containment count (Qₜ) and the recovery probability parameter (δ).

A prospective termination forecast, which is also referred to herein as

$$\frac{dD ( t )}{dt} ,$$

may describe a predicted change in the quantity of an infected but terminated (e.g., deceased) population between a current time period and a future time period. In some embodiments, the prospective recovery termination may be determined based at least in part on the respective containment count (Qₜ) and the termination probability parameter (ρ).

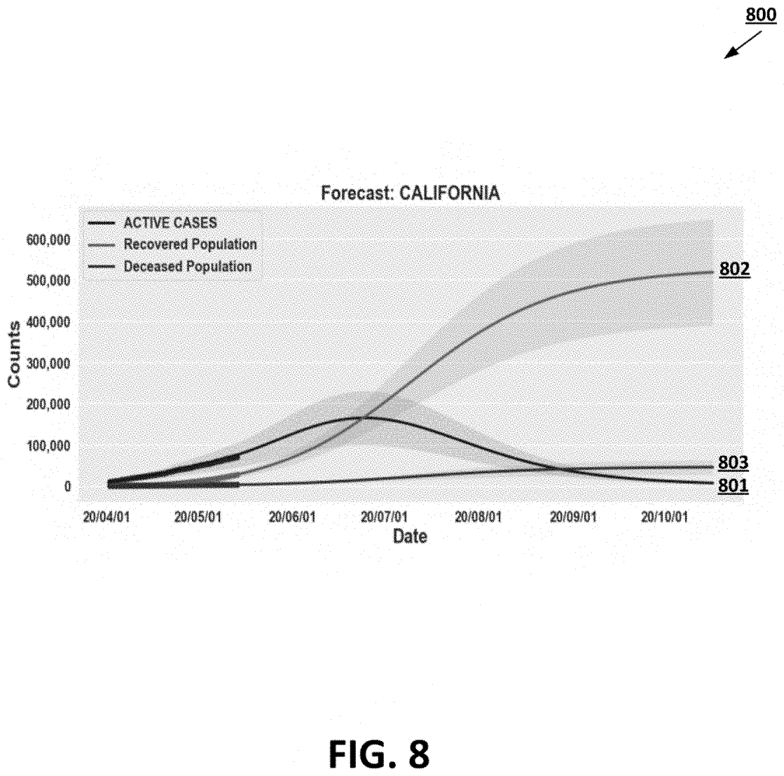

At step/operation 404, the predictive data analysis computing entity 106 performs one or more prediction-based actions based at least in part on the disease spread forecast data object. For example, in some embodiments, the predictive data analysis computing entity 106 generates a predictive output user interface that describes at least some of the data entries described by one or more disease spread forecast data objects. An operational example of such a predictive output user interface 800 is depicted in FIG. 8. As depicted in FIG. 8, the predictive output user interface 800 depicts the forecasted count of active cases 801, recovered cases 802, and deceased cases 803 for the state of California across various time units. In some embodiments, the forecasted count of active cases 801 may be determined based at least in part on prospective infection forecasts for a group of time units, the forecasted count of recovered cases 802 may be determined based at least in part on prospective recovery forecasts for a group of time units, and the forecasted count of deceased cases 803 may be determined based at least in part on prospective termination forecasts for a group of time units.

In some embodiments, the predictive data analysis computing entity 106 may determine one or more population health predictions (e.g., one or more pandemic urgency predictions, one or more medication need predictions, one or more staff need predictions, and/or the like) based at least in part on the inferred predictions and perform one or more prediction-based actions based at least in part on the noted determined population health predictions. Examples of prediction-based actions that may be performed based at least in part on the population health predictions include automated physician notifications, automated patient notifications, automated medical appointment scheduling, automated drug prescription recommendation, automated drug prescription generation, automated implementation of precautionary actions, automated hospital preparation actions, automated insurance workforce management operational management actions, automated insurance server load balancing actions, automated call center preparation actions, automated hospital preparation actions, automated insurance plan pricing actions, automated insurance plan update actions, automated regulatory alert generation actions, and/or the like.

### VI. Conclusion

Many modifications and other embodiments will come to mind to one skilled in the art to which this disclosure pertains having the benefit of the teachings presented in the foregoing descriptions and the associated drawings. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

1. A computer-implemented method for performing optimization-based disease spread forecasting using a multi-risk-level disease spread forecasting machine learning model, the computer-implemented method comprising:
identifying a retrospective timeseries data object;
processing the retrospective timeseries data object to generate a plurality of temporally dynamic parameters of the multi-risk-level disease spread forecasting machine learning model, wherein the plurality of temporally dynamic parameters comprise a general susceptibility parameter, a heightened risk ratio parameter, a heightened risk infection probability parameter, and a non-heightened risk infection probability parameter; and
subsequent to generating the plurality of temporally dynamic parameters, enabling access to the multi-risk-level disease spread forecasting machine learning model to generate a prospective disease spread forecast data object and perform one or more prediction-based actions based at least in part on the prospective disease spread forecast data object.

2. The computer-implemented method of claim 1, wherein:
the prospective disease spread forecast data object describes a prospective termination forecast,
the retrospective timeseries data object describes a retrospective containment count value, and
the prospective termination forecast is determined based at least in part on the retrospective containment count value and a termination probability parameter of the plurality of temporally dynamic parameters.

3. The computer-implemented method of claim 1, wherein:
the prospective disease spread forecast data object describes a prospective recovery forecast,
the retrospective timeseries data object describes a retrospective containment count value, and
the prospective recovery forecast is determined based at least in part on the retrospective containment count value and a recovery probability parameter of the plurality of temporally dynamic parameters.

4. The computer-implemented method of claim 1, wherein:
the prospective disease spread forecast data object describes a prospective containment forecast,
the retrospective timeseries data object describes a retrospective infection count value and a retrospective containment count value, and
the prospective infection forecast is determined based at least in part on the retrospective infection count value, the retrospective containment count value, and a containment probability parameter of the plurality of temporally dynamic parameters.

5. The computer-implemented method of claim 1, wherein:
the prospective disease spread forecast data object describes a prospective infection forecast,
the retrospective timeseries data object describes a retrospective infection count value and a retrospective susceptibility count value, and
the prospective infection forecast is determined based at least in part on: (i) the retrospective infection count value, (ii) the retrospective susceptibility count value, (iii) the heightened risk ratio parameter, (iv) the heightened risk infection probability parameter, (v) the non-heightened risk infection probability parameter, and (vi) the general susceptibility parameter.

6. The computer-implemented method of claim 1, wherein:
the prospective disease spread forecast data object describes a prospective susceptibility forecast,
the retrospective timeseries data object describes a retrospective infection count value and a retrospective susceptibility count value, and
the prospective susceptibility forecast is determined based at least in part on: (i) the retrospective infection count value, (ii) the retrospective susceptibility count value, (iii) the heightened risk ratio parameter, (iv) the heightened risk infection probability parameter, (v) the non-heightened risk infection probability parameter, and (vi) the general susceptibility parameter.

7. The computer-implemented method of claim 6, wherein:
the prospective disease spread forecast data object describes a prospective non-heightened risk forecast,
the retrospective timeseries data object describes a retrospective non-heightened risk count value, and
the prospective non-heightened risk forecast is determined based at least in part on the prospective susceptibility forecast, the retrospective non-heightened risk count value, and the non-heightened risk infection probability parameter.

8. The computer-implemented method of claim 6, wherein:
the prospective disease spread forecast data object describes a prospective heightened risk forecast,
the retrospective timeseries data object describes a retrospective heightened risk count value, and
the prospective heightened risk forecast is determined based at least in part on the prospective susceptibility forecast, the retrospective heightened risk count value, and the heightened risk infection probability parameter.

9. The computer-implemented method of claim 1, wherein determining the general susceptibility parameter comprises:
identifying a plurality of candidate general susceptibility parameter values;
for each candidate general susceptibility parameter value of the plurality of candidate general susceptibility parameter values, generating a mean absolute error measure; and
determining the general susceptibility parameter based at least in part on each mean absolute error measure for a candidate general susceptibility parameter value of the plurality of candidate general susceptibility parameter values.

10. An apparatus for performing optimization-based disease spread forecasting using a multi-risk-level disease spread forecasting machine learning model, the apparatus comprising at least one processor and at least one memory including program code, the at least one memory and the program code configured to, with the processor, cause the apparatus to at least:
identify a retrospective timeseries data object;
process the retrospective timeseries data object to generate a plurality of temporally dynamic parameters of the multi-risk-level disease spread forecasting machine learning model, wherein the plurality of temporally dynamic parameters comprise a general susceptibility parameter, a heightened risk ratio parameter, a heightened risk infection probability parameter, and a non-heightened risk infection probability parameter; and
subsequent to generating the plurality of temporally dynamic parameters, enable access to the multi-risk-level disease spread forecasting machine learning model to generate a prospective disease spread forecast data object and perform one or more prediction-based actions based at least in part on the prospective disease spread forecast data object.

11. The apparatus of claim 10, wherein:
the prospective disease spread forecast data object describes a prospective termination forecast,
the retrospective timeseries data object describes a retrospective containment count value, and
the prospective termination forecast is determined based at least in part on the retrospective containment count value and a termination probability parameter of the plurality of temporally dynamic parameters.

12. The apparatus of claim 10, wherein:
the prospective disease spread forecast data object describes a prospective recovery forecast,
the retrospective timeseries data object describes a retrospective containment count value, and
the prospective recovery forecast is determined based at least in part on the retrospective containment count value and a recovery probability parameter of the plurality of temporally dynamic parameters.

13. The apparatus of claim 10, wherein:
the prospective disease spread forecast data object describes a prospective containment forecast,
the retrospective timeseries data object describes a retrospective infection count value and a retrospective containment count value, and
the prospective infection forecast is determined based at least in part on the retrospective infection count value, the retrospective containment count value, and a containment probability parameter of the plurality of temporally dynamic parameters.

14. The apparatus of claim 10, wherein:
the prospective disease spread forecast data object describes a prospective infection forecast,
the retrospective timeseries data object describes a retrospective infection count value and a retrospective susceptibility count value, and
the prospective infection forecast is determined based at least in part on: (i) the retrospective infection count value, (ii) the retrospective susceptibility count value, (iii) the heightened risk ratio parameter, (iv) the heightened risk infection probability parameter, (v) the non-heightened risk infection probability parameter, and (vi) the general susceptibility parameter.

15. The apparatus of claim 10, wherein:
the prospective disease spread forecast data object describes a prospective susceptibility forecast,
the retrospective timeseries data object describes a retrospective infection count value and a retrospective susceptibility count value, and
the prospective susceptibility forecast is determined based at least in part on: (i) the retrospective infection count value, (ii) the retrospective susceptibility count value, (iii) the heightened risk ratio parameter, (iv) the heightened risk infection probability parameter, (v) the non-heightened risk infection probability parameter, and (vi) the general susceptibility parameter.

16. The apparatus of claim 15, wherein:
the prospective disease spread forecast data object describes a prospective non-heightened risk forecast,
the retrospective timeseries data object describes a retrospective non-heightened risk count value, and
the prospective non-heightened risk forecast is determined based at least in part on the prospective susceptibility forecast, the retrospective non-heightened risk count value, and the non-heightened risk infection probability parameter.

17. The apparatus of claim 15, wherein:
the prospective disease spread forecast data object describes a prospective heightened risk forecast,
the retrospective timeseries data object describes a retrospective heightened risk count value, and
the prospective heightened risk forecast is determined based at least in part on the prospective susceptibility forecast, the retrospective heightened risk count value, and the heightened risk infection probability parameter.

18. The apparatus of claim 10, wherein determining the general susceptibility parameter comprises:
identifying a plurality of candidate general susceptibility parameter values;
for each candidate general susceptibility parameter value of the plurality of candidate general susceptibility parameter values, generating a mean absolute error measure; and
determining the general susceptibility parameter based at least in part on each mean absolute error measure for a candidate general susceptibility parameter value of the plurality of candidate general susceptibility parameter values.

19. A computer program product for performing optimization-based disease spread forecasting using a multi-risk-level disease spread forecasting machine learning model, the computer program product comprising at least one non-transitory computer-readable storage medium having computer-readable program code portions stored therein, the computer-readable program code portions configured to:
identify a retrospective timeseries data object;
process the retrospective timeseries data object to generate a plurality of temporally dynamic parameters of the multi-risk-level disease spread forecasting machine learning model, wherein the plurality of temporally dynamic parameters comprise a general susceptibility parameter, a heightened risk ratio parameter, a heightened risk infection probability parameter, and a non-heightened risk infection probability parameter; and
subsequent to generating the plurality of temporally dynamic parameters, enable access to the multi-risk-level disease spread forecasting machine learning model to generate a prospective disease spread forecast data object and perform one or more prediction-based actions based at least in part on the prospective disease spread forecast data object.

20. The computer program product of claim 19, wherein:
the prospective disease spread forecast data object describes a prospective termination forecast,
the retrospective timeseries data object describes a retrospective containment count value, and
the prospective termination forecast is determined based at least in part on the retrospective containment count value and a termination probability parameter of the plurality of temporally dynamic parameters.
